SEQUENCE LISTING (5 sequences)

SEQ ID NO: 1 - protein, 10 aa, Artificial Sequence
  1133..1142: Target peptide sequence from the C-terminus type XXIII collagen alpha1 chain
SHAHQRTGGN

SEQ ID NO: 2 - protein, 11 aa, Artificial Sequence
  Elongated sequence of SEQ ID NO 1
  SITE 11: Xaa is any amino acid
SHAHQRTGGNX

SEQ ID NO: 3 - protein, 11 aa, Artificial Sequence
  Elongated sequence of SEQ ID NO 1
SHAHQRTGGNA

SEQ ID NO: 4 - protein, 9 aa, Artificial Sequence
  Truncated sequence of SEQ ID NO 1
SHAHQRTGG

SEQ ID NO: 5 - protein, 10 aa, Artificial Sequence
  Non-sense standard peptide sequence
GVAPGIGPGG

(19) United States
(12) Patent Application Publication
(10) Pub. No.: US 2024/0210405 A1
(43) Pub. Date: Jun. 27, 2024

(54) Type XIX Collagen Assay

(71) Applicant: Nordic Bioscience A/S, Herlev (DK)

(72) Inventors: Nicholas Willumsen, Dyssegard (DK); Jeppe Thorlacius-Ussing, Herlev (DK)

(73) Assignee: Nordic Bioscience A/S, Herlev (DK)

(21) Appl. No.: 17/914,101

(22) PCT Filed: Mar. 22, 2021
(86) PCT No.: PCT/EP2021/057319

§ 371 Date: Sep. 23, 2022

(30) Foreign Application Priority Data
Mar. 23, 2020 (GB) 2004195.0

(51) Int. Cl. G01N 33/574; C07K 16/18; G01N 33/68

(52) U.S. Cl. CPC G01N 33/57488; C07K 16/18; G01N 33/6887; C07K 2317/34; G01N 2333/78; G01N 2470/10

(57) ABSTRACT

The present invention relates to monoclonal antibodies that target collagen type XIX, and to immunoassays and kits employing the antibodies. The assays of the invention can be used in the diagnosis and monitoring of cancer.

### FIELD OF THE INVENTION

The present invention relates to monoclonal antibodies that target collagen type XIX, and to immunoassays and kits employing said antibodies.

### INTRODUCTION

Type XIX collagen is a poorly characterized collagen associated with the basement membrane. It has shown altered regulation during breast cancer progression and the NC1 (XIX) domain has shown anti-tumorigenic signalling properties. However, little is known about the biomarker potential of collagen XIX in cancer.

Lung cancer is the most commonly diagnosed cancer and is the leading cause of cancer death (1). Non-small cell lung cancer (NSCLC) represents approximately 85% of all lung cancer cases, wherein adenocarcinoma (AC) and squamous cell carcinoma (SCC) are the most common subtypes (2,3). The majority of lung cancer cases are diagnosed in later stages, resulting in a grim overall five-year survival of 19% (4). However, for patients diagnosed in the localized stages, where most of the patients can benefit from surgical resection, the five-year survival is 56% (2,4). Early detection is therefore one of the primary ways to improve survival for lung cancer patients.

The tumour microenvironment is intricately connected to the traditional hallmarks that dictate cancer progression 5. One of the major components of the tumour microenvironment is the extracellular matrix (ECM), the non-cellular part of tissues, which influences virtually all of these hallmarks (6. The most dominant proteins in the ECM are the collagens, of which there are 28 different types (7). Type XIX collagen is a minor collagen that comprises three α1 (XIX) chains forming a 400 kDa homotrimer. Each chain comprises five collagenous triple helix domains interspersed with six non-collagenous domains. Based on its primary sequence, type XIX collagen belongs to the Fibril-Associated Collagen with Interrupted Triple helices family of collagens which mediate interactions between fibrillar collagens and other ECM components (8-10).

Collagen XIX expression is widespread in developing mice but more restricted in adults, where it mostly accumulates in brain tissue (11). In human adults, expression is found in brain, skeletal muscle, spleen, prostate, kidney, liver, placenta, colon, skin and breast tissue (12,13). Although the function of collagen XIX is not fully understood, it does seem clear that it has a developmental role. Collagen XIX is involved in embryonic muscle differentiation and oesophagus development (10,14,15). Collagen XIX is also involved in the formation of synapses in the hippocampus and in the formation of axons in spinal cord neurons (16,17). An overexpression of collagen XIX in muscle of amyotrophic lateral sclerosis (ALS) patients has also been described and was associated with worse prognosis (18,19).

Overall, tissue localization of collagen XIX protein is mostly associated with the vascular, neural, muscular and some epithelial basement membrane zones (BMZ)s (12). Interestingly, protein staining of type XIX collagen in the epithelial BMZ of breast carcinoma is partially lost in localized ductal carcinoma and is completely absent in invasive carcinomas. This loss occurs earlier than the loss of type IV collagen and laminin, suggesting that decreased collagen XIX levels is a result of the early stages of BMZ remodelling in pre-invasive tumours (13).

Similar to the release of matrikines from the NC1 domains of type IV, XV and XVIII collagens, the C-terminal NC1 domain of collagen XIX can be cleaved off and released. The resulting peptide can inhibit the growth and angiogenesis of melanoma in vivo and inhibit invasiveness in vitro (20). The NC1 domain is cleaved off by the plasmin protease and interacts with αvβ3 integrin to inhibit the FAK/PI3K/Akt/mTOR signalling pathway as well as inhibit GSK3B phosphorylation (21,22). Interestingly, the NC1 domain also triggers the formation of inhibitory nerve terminals, although it interacts with a different integrin receptor, α5β1, when doing so (23).

### SUMMARY OF THE INVENTION

The present inventors have developed a monoclonal antibody that specifically recognises Type XIX collagen, specifically the C terminus of the α1 chain; and an immunoassay, in particular an enzyme-linked immunosorbent assay (ELISA) to detect type XIX collagen in a biofluid sample. The inventors have determined that Type XIX collagen can be used as a biomarker for detection of cancer. In particular PRO-C19 is particularly good at discriminating between NSCLC and healthy controls and can be used as a biomarker for early detection of NSCLC.

Accordingly, in a first aspect the present invention relates to a monoclonal antibody that specifically recognises and binds to the C-terminus of type XIX collagen α1 chain (also referred to herein as the target peptide), the C-terminus having the amino acid sequence SHAHQRTGGN (SEQ. ID No. 1) (also referred to herein as the target sequence).

Preferably, the monoclonal antibody is a monoclonal antibody that has been raised against a synthetic peptide having the C-terminus amino acid sequence SHAHQRTGGN (SEQ. ID No. 1). The synthetic peptide used to raise the antibody may be a synthetic peptide linked at its N-terminus to a carrier protein. Exemplary carrier proteins include proteins such as, but not limited to, keyhole limpet hemocyanin (KLH). The synthetic peptide may be linked to the carrier protein via any suitable linkage, which may include one or more additional amino acid residues at the N-terminus of the peptide. The monoclonal antibody may have been raised via suitable techniques known those skilled in the art such as, but not limited to, immunizing a mouse or other mammal, isolating and fusing spleen cells from the immunized mammal with hybridoma cells, and then culturing the resultant hybridoma cells to secure monoclonal growth.

In a preferred embodiment, the monoclonal antibody does not specifically recognise or bind to a peptide having the C-terminus amino acid sequence SHAHQRTGGNX (SEQ. ID No. 2), wherein X represents any amino acid. Thus, the monoclonal antibody preferably does not specifically recognise or bind to elongated variants of the target peptide in which the target amino acid sequence has been extended at the C-terminus by one or more amino acids. Preferably, the monoclonal antibody preferably does not specifically recognise or bind to a peptide having the C-terminus amino acid sequence SHAHQRTGGNA (SEQ. ID No. 3).

In a preferred embodiment, the monoclonal antibody does not specifically recognise or bind to a peptide having the C-terminus amino acid sequence SHAHQRTGG (SEQ. ID No. 4). Thus, the monoclonal antibody preferably does not specifically recognise or bind to shortened variants of the target peptide in which the target amino acid sequence has been truncated at the C-terminus by one or more amino acids.

In a preferred embodiment, the monoclonal antibody does not specifically recognise or bind to a peptide having the C-terminus amino acid sequence GVAPGIGPGG (SEQ. ID No. 5). Thus, the monoclonal antibody preferably does not specifically recognise or bind to a non-sense standard peptide.

In a second aspect, the present invention relates to a method of immunoassay for detecting type XIX collagen in a human biofluid sample, said method comprising contacting a human biofluid sample with a monoclonal antibody according to the first aspect of the invention, and detecting binding between the monoclonal antibody and peptides in the sample.

Preferably, the detection is quantitative. Thus, the method may comprise detecting and determining the amount of binding between the monoclonal antibody and peptides in the sample.

Preferably, the immunoassay is a competitive immunoassay.

Preferably, the immunoassay is an enzyme-linked immunosorbent assay (ELISA). Preferably the ELISA is a competitive ELISA.

The human biofluid sample may be for instance blood, serum, plasma or urine. Preferably the sample is serum or plasma.

The human biofluid sample may be a sample from a human patient having medical signs or symptoms indicative of cancer. Preferably the biofluid sample is a sample from a human patient having medical signs or symptoms indicative of pancreatic, colo-rectal, kidney, stomach (gastric), ovarian, breast, bladder, lung, head and neck, prostate, or liver cancer or melanoma, preferably breast, lung or ovarian cancer, in particular lung cancer, especially Non-small cell lung cancer (NSCLC).

The method may be an immunoassay method for diagnosing and/or monitoring and/or assessing the likelihood of cancer in a patient, the method comprising contacting a biofluid sample obtained from said patient with the monoclonal antibody, detecting and determining the amount of binding between the monoclonal antibody and peptides in the sample, and correlating said amount of binding with values associated with normal healthy subjects and/or values associated with known disease severity and/or values obtained from said patient at a previous time point. Preferably the cancer is pancreatic, colo-rectal, kidney, stomach (gastric), ovarian, breast, bladder, lung, head and neck, prostate, or liver cancer or melanoma, more preferably breast, lung or ovarian cancer, in particular lung cancer, especially Non-small cell lung cancer (NSCLC).

In some embodiments of the method according to the second aspect, the amount of binding of the monoclonal antibody specific for the epitope of type XIX collagen peptide with C-terminal amino acid sequence SHAHQRTGGN (SEQ ID No. 1) is correlated with one or more predetermined cut-off values.

As used herein the “cut-off value” means an amount of binding that is determined statistically to be indicative of a high likelihood of a subject having cancer. The measured value of biomarker binding in a patient sample that is at or above the statistical cutoff value may correspond to at least a 70% probability, preferably at least an 80% probability, preferably at least an 85% probability, more preferably at least a 90% probability, and most preferably at least a 95% probability of the presence or likelihood of cancer. The “cut-off value” can be calculated by comparing the results obtained from patients who have been diagnosed with a cancer and healthy controls.

The predetermined cut-off value for the amount of binding of the monoclonal antibody specific for the C-terminal amino acid sequence SHAHQRTGGN (SEQ ID No. 1) may be within the range 50.0-200.0 ng/ml. Preferably the predetermined cut-off value for the amount of binding of the monoclonal antibody specific for the C-terminal amino acid sequence SHAHQRTGGN (SEQ ID No. 1) is in the range 75.0-150.0 ng/ml, more preferably in the range 90.0-120.0 ng/ml, most preferably at least 118.9 ng/ml. In this regard, through the use of statistical analyses it has been found that a measured amount of binding of the monoclonal antibody specific for the C-terminal amino acid sequence SHAHQRTGGN (SEQ ID No. 1) in the range 50.0-200.0 ng/ml, in particular of at least 118.9 ng/ml or greater may be determinative of a patient likely to have cancer, in particular lung cancer, such as NSCLC. By having a statistical cut-off value in the range 50.0-200.0 ng/ml, in particular of at least 118.9 ng/ml it is possible to utilise the method of the invention to give a prediction of a cancer diagnosis with a high level of confidence. In particular, a value in the range 50.0-200.0 ng/ml, in particular at least 118.9 ng/ml or greater may be determinative of a patient with NSCLC. Applying such statistical cut-off values are particularly advantageous as it results in a standalone diagnostic assay; i.e. it removes the need for any direct comparisons with healthy individuals and/or patients known to have cancer in order to arrive at a diagnostic conclusion. An expedited conclusive prediction may result in the patients being treated at an earlier stage, which may in turn improve overall chances of survival, and/or reduce the risk of hospitalisation.

In patients who have been diagnosed with lung cancer, in particular NSCLC, a predetermined cut-off value for the amount of binding of the monoclonal antibody specific for the C-terminal amino acid sequence SHAHQRTGGN (SEQ ID No. 1) can be used to provide an indication of the stage of the cancer. The predetermined cut-off value for the amount of binding of the monoclonal antibody specific for the C-terminal amino acid sequence SHAHQRTGGN (SEQ ID No. 1) can be in the range 40.0-75.0 ng/ml, preferably at least 55.6 ng/ml. In this regard, through the use of statistical analyses it has been found that a measured amount of binding of the monoclonal antibody specific for the C-terminal amino acid sequence SHAHQRTGGN (SEQ ID No. 1) in the range of 40.0-75.0 ng/ml, preferably of at least 55.6 ng/ml or greater may be determinative of a patient likely to have late stage cancer, in Stage III or stage IV NSCLC. By having a statistical cut-off value in the range of 40.0-75.0 ng/ml, in particular of at least 55.6 ng/ml it is possible to utilise the method of the invention to give a prediction of a cancer stage and progression with a high level of confidence. In particular, a value in the range of 40.0-75.0 ng/mL, preferably of at least 55.6 ng/ml or greater may be determinative of a patient with at least Stage III NSCLC. An expedited conclusive prediction may assist in the monitoring of progression of the cancer in patients, and the monitoring of the effectiveness of treatment. The use of a cut-off value may assist in identifying if a treatment regime is ineffective, and the cancer progressing, so that alternative treatment can be sought at an earlier stage, which may in turn improve overall chances of survival.

In a third aspect, the present invention relates to an assay kit comprising a monoclonal antibody according to the first aspect of the invention, and at least one of:
a streptavidin coated well plate;
a N-terminal biotinylated peptide having the C-terminus amino acid sequence SHAHQRTGGN (SEQ. ID No. 1); and
a calibrator peptide having the C-terminus amino acid sequence SHAHQRTGGN (SEQ. ID No. 1).

The kit may be for use in diagnosing or predicting the risk of cancer preferably in conjunction with the methods according to the second aspect of the invention. Preferably the cancer is pancreatic, colo-rectal, kidney, stomach (gastric), ovarian, breast, bladder, lung, head and neck, prostate, or liver cancer or melanoma, more preferably breast, lung or ovarian cancer, in particular lung cancer, especially Non-small cell lung cancer (NSCLC).

### Definitions

As used herein, the terms “peptide” and “polypeptide” are used synonymously.

As used herein the term “monoclonal antibody” refers to both whole antibodies and to fragments thereof that retain the binding specificity of the whole antibody, such as for example a Fab fragment, Fv fragment, or other such fragments known to those skilled in the art. Antibodies which retain the same binding specificity may contain the same complementarity-determining regions (CDR). The CDR of an antibody can be determined using methods known in the art such as that described by Kabat et al. (38) Antibodies can be generated from B cell clones as described in the examples. The isotype of the antibody can be determined by ELISA specific for human IgM, IgG or IgA isotype, or human IgG1, IgG2, IgG3 or IgG4 subclasses. Other suitable methods can be used to identify the isotype.

The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example, primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lambda chain together with primers which anneal to the V kappa or V lambda leader sequence. The full length heavy and light chains can be generated and sequenced.

As used herein the term “C-terminus” refers to the extremity of a polypeptide, i.e. at the C-terminal end of the polypeptide, and is not to be construed as meaning in the general direction thereof. Likewise, the term “N-terminus” refers to the extremity of a polypeptide, i.e. at the N-terminal end of the polypeptide, and is not to be construed as meaning in the general direction thereof.

As used herein the term, the term “competitive immunoassay” refers to an immunoassay in which the target peptide present in a sample (if any) competes with known amount of target of peptide (which, for example, is bound to a fixed substrate or is labelled) for binding to an antibody, which is a technique known to those skilled in the art.

As used herein the term “ELISA” (enzyme-linked immunosorbent assay) refers to an immunoassay in which the target peptide present in a sample (if any) is detected using antibodies linked to an enzyme, such as horseradish peroxidase or alkaline phosphatase. The activity of the enzyme is then assessed by incubation with a substrate generating a measurable product. The presence and/or amount of target peptide in a sample can thereby be detected and/or quantified. ELISA is a technique known to those skilled in the art.

As used herein the term “amount of binding” refers to the quantification of binding between monoclonal antibody and target peptide, which said quantification is determined by comparing the measured values of target peptide in the biofluid samples against a calibration curve, wherein the calibration curve is produced using standard samples of known concentration of the target peptide. In the specific assay disclosed herein which measures in biofluids target peptides having the C-terminus amino acid sequence SHAHQRTGGN (SEQ. ID No. 1), the calibration curve is produced using standard samples of known concentration of a calibration peptide having the C-terminus amino acid sequence SHAHQRTGGN (SEQ. ID No. 1, (and which may in particular consist of the amino acid sequence SHAHQRTGGN (SEQ. ID No. 1)). The values measured in the biofluid samples are compared to the calibration curve to determine the actual quantity of target peptide in the sample.

As used herein, the term “PRO-C19” refers to type XIX collagen having the C-terminal amino acid sequence SHAHQRTGGN (SEQ. ID No. 1).

The invention will be demonstrated in the examples below which refer to the following figures:

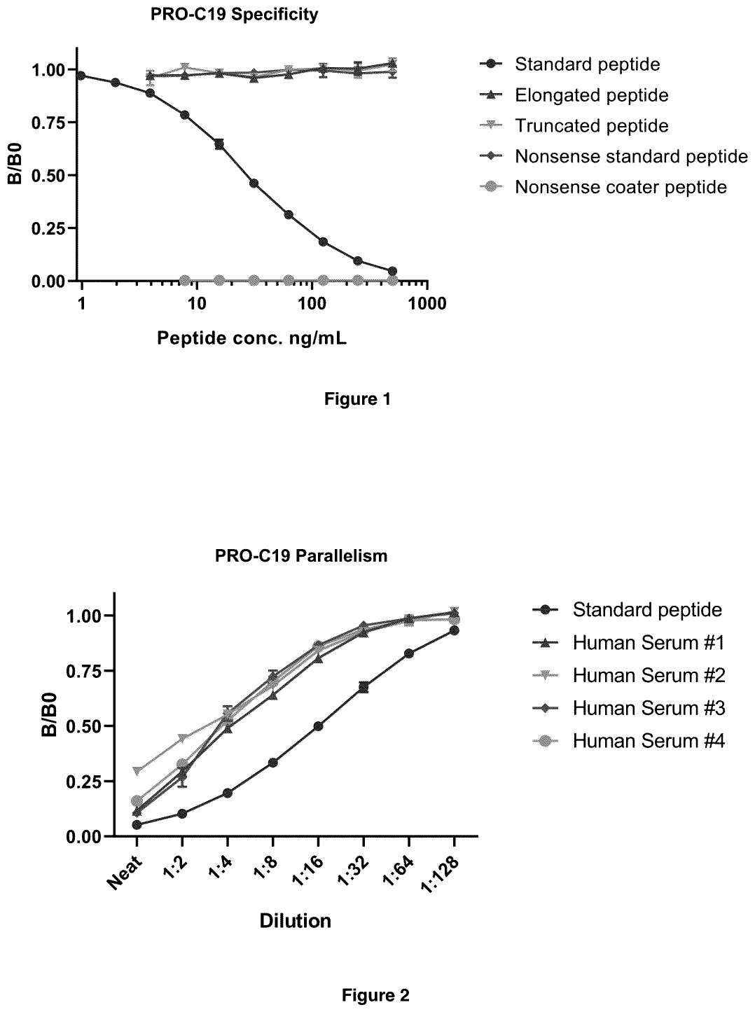

FIG. 1 shows PRO-C19 assay specificity. Inhibition curve for the standard peptide (SHAHQRTGGN SEQ. ID No. 1), elongated peptide (SHAHQRTGGNA SEQ. ID No. 3), truncated peptide (SHAHQRTGG SEQ. ID No. 4) as well as non-sense standard peptide (GVAPGIGPGG SEQ. ID No. 5) and a non-sense coater peptide (Biotin-GVAPGIGPGG). Peptides were diluted twofold in series to evaluate their propensity to compete for antibody binding. Signal is shown as a fraction of the background absorbance (B0), corresponding to assay buffer, as a function of the peptide concentration on a logarithmic scale. Error bars indicate standard deviation from duplicate measurements.

FIG. 2 shows PRO-C19 parallelism. Inhibition curve for the standard peptide and human serum samples diluted twofold to assess linearity of dilution or parallelism. Signal is shown as a fraction of the background absorbance (B0), corresponding to assay buffer, as a function of the dilution steps. Error bars indicate standard deviation from duplicate measurements.

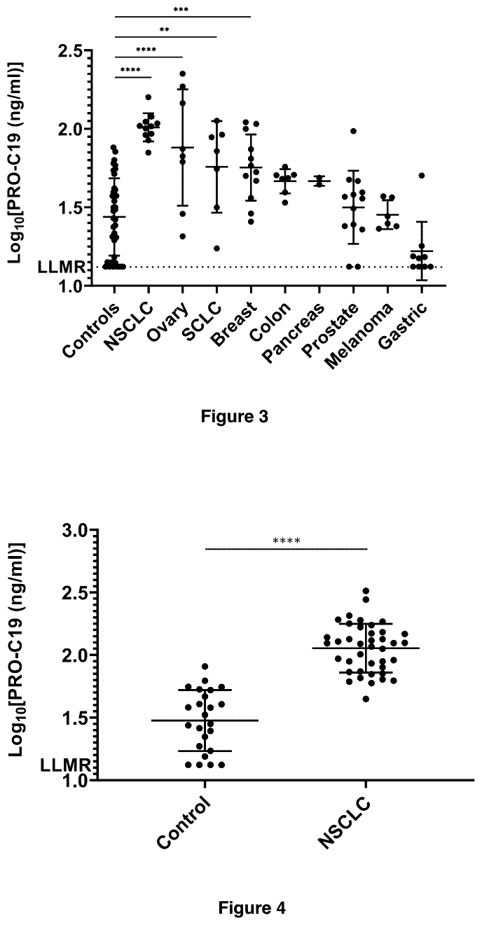

FIG. 3 shows PRO-C19 in cohort 1. Quantification of PRO-C19 in the serum of healthy controls (n=38) and a range of cancer types including NSCLC (n=11), ovary (n=8), SCLC (n=7), breast (n=12), colon (n=7), pancreas (n=2), prostate (n=13), melanoma (n=6), gastric (n=9). PRO-C19 levels were log-transformed and are presented with mean and standard deviation. Samples measuring below the LLMR were given the value of the LLMR, as determined in the validation of PRO-C19. Differences in PRO-C19 levels were evaluated by ordinary one-way ANOVA corrected for multiple comparisons with Dunnett test. **** indicates a p-value below 0.0001. *** indicates a p-value below 0.001. ** indicates a p-value below 0.01.

FIG. 4 shows PRO-C19 in cohort 2. Quantification of PRO-C19 in the serum of healthy controls (n=24) and NSCLC (n=40). PRO-C19 levels were log-transformed and are presented with mean and standard deviation. Samples measuring below the LLMR were given the value of the LLMR, as determined in the validation of PRO-C19. Differences in PRO-C19 levels were evaluated by two-tailed unpaired t-test. **** indicates a p-value below 0.0001.

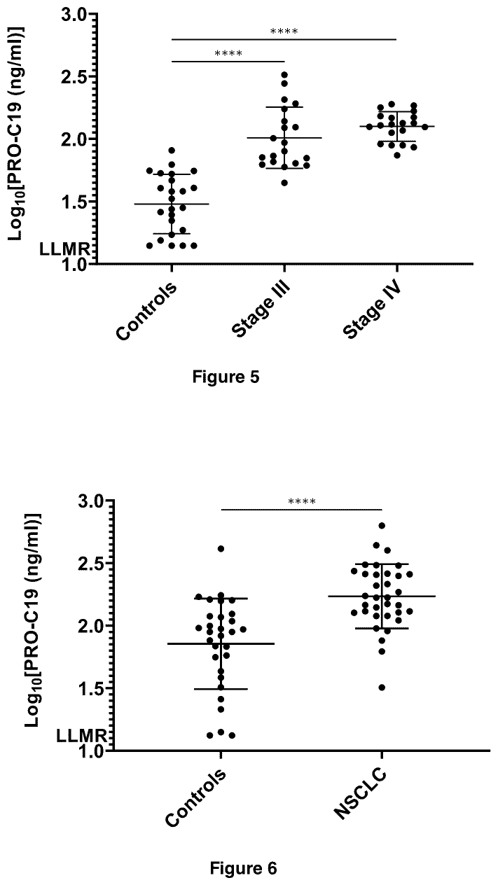

FIG. 5 shows PRO-C19 in stages of cohort 2. Quantification of PRO-C19 in the serum of healthy controls (n=24) and NSCLC stage III (n=20) and stage IV (n=20). PRO-C19 levels were log-transformed and are presented with mean and standard deviation. Samples measuring below the LLMR were given the value of the LLMR, as determined in the validation of PRO-C19. Differences in PRO-C19 levels were evaluated by ordinary one-way ANOVA corrected for multiple comparisons with Dunnett test. **** indicates a p-value below 0.0001.

FIG. 6 shows PRO-C19 in cohort 3. Quantification of PRO-C19 in the serum of healthy controls (n=30) and NSCLC (n=34). PRO-C19 levels were log-transformed and are presented with mean and standard deviation. Samples measuring below the LLMR were given the value of the LLMR, as determined in the validation of PRO-C19. Differences in PRO-C19 levels were evaluated by two-tailed unpaired t-test. **** indicates a p-value below 0.0001.

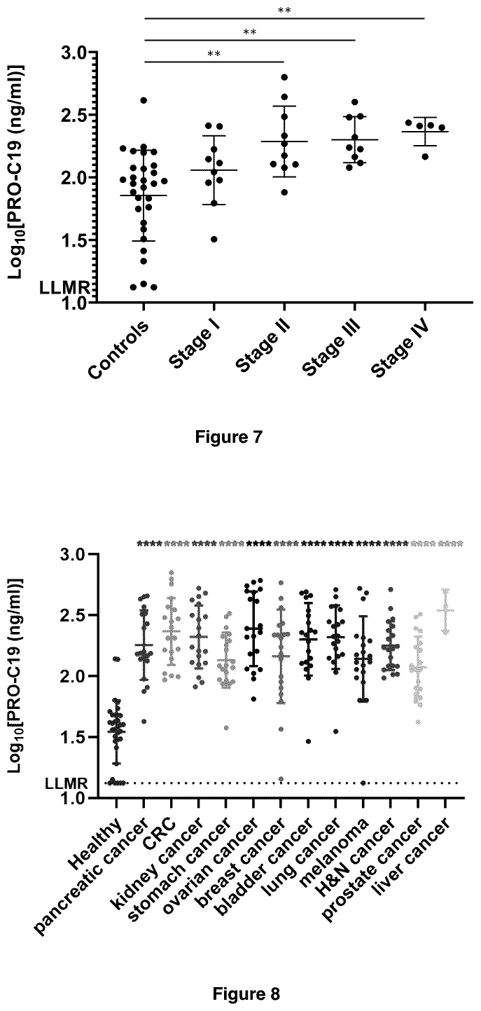

FIG. 7 shows PRO-C19 in stages of cohort 3. Quantification of PRO-C19 in the serum of healthy controls (n=30) and NSCLC stage I (n=10), stage II (n=10), stage III (n=9) and stage IV (n=5). PRO-C19 levels were log-transformed and are presented with mean and standard deviation. Samples measuring below the LLMR were given the value of the LLMR, as determined in the validation of PRO-C19. Differences in PRO-C19 levels were evaluated by ordinary one-way ANOVA corrected for multiple comparisons with Dunnett test. ** indicates a p-value below 0.01.

FIG. 8 shows PRO-C19 in cohort 4. Quantification of PRO-C19 in the serum of healthy controls (n=33) and pancreatic cancer (n=20), colorectal cancer (CRC, n=20), kidney cancer (n=20), stomach cancer (n=20), ovarian cancer (n=20), breast cancer (n=20), bladder cancer (n=20), lung cancer (n=20), melanoma (n=20), head and neck cancer (H&N, n=20), prostate cancer (n=20) and liver cancer (n=3). PRO-C19 levels were log-transformed and are presented with mean and standard deviation. Samples measuring below the LLMR were given the value of the LLMR, as determined in the validation of PRO-C19. Differences in PRO-C19 levels between cancers and healthy controls were evaluated by ordinary one-way ANOVA corrected for multiple comparisons with Dunnett test. **** indicates a p-value below 0.0001 for the comparisons between cancers and healthy controls.

### METHODS

### Pro-C19 ELISA Protocol:

The ten amino acid peptide ₁₁₃₃SHAHQRTGGN₁₁₄₂ (SEQ. ID No. 1), found in the C-terminus of type XIX collagen (UniProtKB: Q14993), was purchased from Genscript (Piscataway, NJ, USA) and used for immunization. The production of monoclonal antibodies has been described elsewhere (24). Several optimizations were made to the ELISA including the choice of assay buffer, incubation time and temperature as well as concentrations of antibody and peptides. The final PRO-C19 protocol was performed as follows: a 96-well streptavidin-coated ELISA plate was coated with 100 μl/well of 2.5 ng/ml biotinylated SHAHQRTGGN peptide dissolved in assay buffer (25 mM TBS, 1% BSA (w/v), 0.1% Tween-20 (w/v), 2 g/l NaCl, pH 8.0) and incubated for 30 minutes at 20° ° C. with shaking at 300 RPM. After washing five times with washing buffer (25 mM Tris, 50 mM NaCl, pH 7.2), 20 μl/well of sample was added in duplicates followed by 100 μl/well of 60 ng/ml HRP-labelled monoclonal antibody in assay buffer and incubated for 1 hour at 20° C. with shaking at 300 RPM. After a second washing cycle, 100 μl/well of TMB was added and incubated 15 minutes in darkness at 20° C. with shaking at 300 RPM. The reaction was stopped by adding 100 μl/well of 1% H₂SO₄. Absorbance was measured at 450 nm with 650 nm as reference. To generate a standard curve, 20 μl/well of 500 ng/ml SHAHQRTGGN (SEQ. ID No. 1) peptide, serially diluted twofold, was added to appropriate wells and a four-parametric mathematical fit was used to generate the curve. Each plate included 5 quality control samples comprising one human serum, one horse serum, one bovine cartilage explant and two peptide-in-assay-buffer samples to monitor intra- and inter-assay variation.

### Technical Validation of the PRO-C19 ELISA:

Antibody specificity was tested by the inhibition of signal by twofold dilutions of the standard peptide (SHAHQRTGGN—SEQ. ID No. 1), elongated peptide (SHAHQRTGGNA—SEQ. ID No. 3), truncated peptide (SHAHQRTGG—SEQ. ID No. 4) as well as non-sense standard peptide (GVAPGIGPGG—SEQ. ID No. 5) and a non-sense coater peptide (Biotin-GVAPGIGPGG). Linearity or parallelism was tested by serially diluting human serum samples twofold and calculating the percentage recovery relative to the dilution. Accuracy was tested by spiking the standard peptide into a human serum sample and calculating the percentage recovery of the peptide in the spiked sample. Influence of commonly interfering substances including haemoglobin, lipids and biotin was evaluated in human serum spiked with either high or low concentrations of the interfering agents (haemoglobin low=2.5 mg/ml, high=5 mg/ml; lipid low=1.5 mg/ml, high=5 mg/ml; biotin low=3 ng/ml, high=9 ng/ml). Assay interference was calculated as the percentage recovery of the spiked sample relative to the non-spiked sample. Assay variation was tested by ten independent runs using ten quality control samples run in double-determinations. Five of the quality control samples were human serum, one was horse serum, one was bovine cartilage explant and three were standard peptide in assay buffer of varying concentrations. Intra-assay variation was calculated as the mean coefficient of variance (CV %) for the double determinations of each of the ten runs. Inter-assay variation was calculated as the overall CV % across the ten runs. Lower- and upper-limit of measurement range (LLMR and ULMR, respectively) were determined across the ten independent runs and denotes the boundaries of the linear range of the standard curve. Analyte stability was determined for three human serum samples incubated at 4 or 20° C. for 2, 4, 24 or 48 hours. Stability was calculated as the percentage recovery of the incubated sample relative to the control sample kept at −20° C. Freeze-thaw stability was evaluated by freezing and thawing human serum samples up to 4 cycles. Stability was calculated as the percentage recovery of the thawed sample relative to the sample that underwent a single freeze-thaw cycle. Lower limit of detection was calculated as the mean concentration of 21 blank samples containing assay buffer with 3 standard deviations added. Upper limit of detection was calculated as the mean concentration of standard peptide corresponding to the highest concentration of the standard curve across the ten independent runs with 3 standard deviations subtracted.

### Patient Samples:

The first cohort was in part obtained from the commercial vendor Asterand Bioscience (Detroit, MI, USA). It included serum from 75 cancer patients including breast (n=12), colon (n=7), gastric (n=9), melanoma (n=6), NSCLC (n=11), ovary (n=8), pancreas (n=2), prostate (n=13), small cell lung cancer (SCLC) (n=7) along with 38 healthy controls from the commercial vendor Valley Biomedical (Winchester, VA, USA).

The second and third cohort was obtained from the commercial vendor Proteogenex (Los Angeles, CA, USA). The second cohort included 40 patients with NSCLC of which 20 were in stage III and 20 were stage IV. It also included 24 healthy controls obtained from Valley Biomedical. The third cohort included 34 NSCLC patients of which 10 were stage I, 10 stage II, 9 stage III and 5 stage IV. It also included 30 healthy controls obtained from Proteogenex and Valley Biomedical.

The fourth cohort included 20 patients each of pancreatic-, colorectal-, kidney-, stomach-, ovarian-, breast-, bladder-, lung-, melanoma-, head and neck- and prostate-cancer. It also included 3 liver cancer patients and 33 healthy controls. All cancer samples were obtained from Proteogenex and the healthy controls were obtained from BioIVT (Westbury, NY, USA).

According to the vendors, sample collection was approved by an Institutional Review Board or Independent Ethical Committee and patients gave their informed consent. All investigations were carried out according to the Helsinki Declaration.

### Statistics:

PRO-C19 levels were log transformed and tested for normality by D'Agostino-Pearson omnibus test. Comparison of PRO-C19 levels between healthy and NSCLC and PRO-C19 levels between NSCLC subtypes was done using unpaired, two-tailed t-test. Comparison of PRO-C19 levels across several groups was done using ordinary one-way ANOVA corrected for multiple comparisons using Dunnett test. Differences in age between groups was evaluated using unpaired, two-tailed t-test. Differences in gender and ethnicity was evaluated using Fisher's exact test. The correlation between PRO-C19 levels and BMI, age, smoking and date of sample collection was evaluated using linear regression. Diagnostic accuracy was tested by the area under the receiver operating characteristics (AUROC) curve. Sensitivity and specificity were determined at the estimated optimal cut-off value according to the Youden Index. A p value below 0.05 was considered significant. Asterisks indicate the following significance levels: *p<0.05; **p<0.01; ***p<0.001; ****p<0.0001. When doing multiple comparisons tests, multiplicity adjusted p-values are reported. Statistical analysis and graphs were done in GraphPad Prism (version 8.2 for Windows, GraphPad Software, San Diego, California USA, www.graphpad.com) and MedCalc (MedCalc Statistical Software version 18.11.6 (MedCalc Software bvba, Ostend, Belgium; https://www.medcalc.org; 2019).

### Results

Specificity of the PRO-C19 assay was assessed by the proficiency of peptides to compete for binding to the monoclonal antibody. Peptides that were tested included the standard peptide (SHAHQRTGGN—SEQ. ID No. 1), elongated peptide (SHAHQRTGGNA—SEQ. ID No. 3), truncated peptide (SHAHQRTGG—SEQ. ID No. 4), non-sense peptide (GVAPGIGPGG—SEQ. ID No. 5) and a non-sense coater peptide (Biotin-GVAPGIGPGG). Only the standard peptide dose-dependently inhibited the signal (FIG. 1 shows PRO-C19 assay specificity). The non-sense coater peptide resulted in no detectable signal. In all, this indicates that the assay is specific to the SHAHQRTGGN (SEQ. ID No. 1) epitope of type XIX collagen.

Technical validation of the PRO-C19 assay is summarized in Table 1. Linearity of dilution and parallelism was acceptable once serum samples were diluted 1:4, after which there was an average dilution recovery of 101.7% (FIG. 2). Matrix accuracy in serum was acceptable with an average spiking recovery of 118.6% using the standard peptide spiked into human serum samples at a final dilution of 1:4. The influence of commonly interfering agents including haemoglobin, lipids and biotin was not observed. Inter-assay variation was 10.9% and intra-assay variation was 6.6%. The measurement range was determined as 3.31-214.3 ng/ml and the limits of detection as 1.23-443.5 ng/ml. Analyte stability was acceptable for up to 24 hours at 4° C. and up to 4 hours at 20° C. Freeze-thaw stability was acceptable for over 4 freeze-thaw cycles.

To explore the usefulness of PRO-C19 in the cancer context, PRO-C19 was assessed in a cohort consisting of a range of cancer types including 12 breast cancer samples, 7 colon cancer, 9 gastric cancer, 6 melanoma, 11 NSCLC, 8 ovarian cancer, 2 pancreatic cancer, 13 prostate cancer, 7 SCLC as well as 38 healthy controls (Table 2). In the cancer group there was no significant association between PRO-C19 levels and age, BMI or smoking history. PRO-C19 levels were significantly elevated in NSCLC (p<0.0001), SCLC (p=0.0081), breast (p=0.0005) and ovarian cancer (p<0.0001) (FIG. 3). Although not significant, colon and pancreatic cancer groups also had higher mean PRO-C19 levels whereas gastric cancer had lower mean PRO-C19 levels as compared to healthy controls. With a cut-off of 63.3 ng/ml, PRO-C19 could discriminate between healthy and NSCLC with an AUROC of 0.995 with a corresponding sensitivity of 100% and specificity of 94.74% (Table 5). PRO-C19 could also discriminate between healthy and SCLC with an AUROC of 0.808 at a cut-off of 54.3 ng/ml with a corresponding sensitivity of 71.4% and specificity of 84.2%. PRO-C19 could also discriminate between healthy and breast cancer with an AUROC of 0.814 at a cut-off of 41.85 ng/ml with a corresponding sensitivity of 75% and specificity of 78.9%. Lastly, PRO-C19 could also discriminate between healthy and ovarian cancer with an AUROC of 0.839 at a cut-off of 60.31 ng/ml with a corresponding sensitivity of 75% and specificity of 92.1%. Overall, it seems that circulation levels of collagen XIX are elevated in several different cancer types. The role of PRO-C19 in NSCLC was then explored.

PRO-C19 was assessed in a cohort of NSCLC patients including 20 stage III and 20 stage IV patients as well as 24 healthy controls (Table 3). The control group was significantly younger, had a smaller proportion of males and a smaller proportion of Caucasian ethnicity compared to the NSCLC group. Within the NSCLC group itself, there was no significant association between PRO-C19 levels and the date of sample collection, gender, age, BMI, smoking history, tumour grade or histological subtype (AC and SCC). Mean PRO-C19 levels significantly elevated (p<0.0001) up to 3.5-fold higher for the NSCLC group compared to controls (FIG. 4). At a cut-off of 55.6 ng/ml, PRO-C19 could discriminate between healthy and NSCLC with an AUROC of 0.980 with a corresponding sensitivity of 97.5% and a specificity of 91.67% (Table 5). Divvied up into stages III and IV, PRO-C19 levels for each stage were also significantly elevated compared to healthy controls (p<0.0001) (FIG. 5). These results confirm that PRO-C19 levels are elevated in the circulation of NSCLC patients.

Next, the use of PRO-C19 in earlier stages of NSCLC was explored. To this end, PRO-C19 was assessed in a separate cohort of NSCLC patients including 10 stage I, 10 stage II, 9 stage III, 5 stage IV as well as 30 healthy controls (Table 4). The control group was significantly younger and had a smaller proportion of Caucasians as compared to the NSCLC group. There was no significant difference in gender between the two groups. Within the NSCLC group, there was no significant association with date of sample collection, gender, age, BMI, smoking history, tumour grade or histological subtype (AC or SCC). Mean PRO-C19 levels were significantly elevated (p<0.0001) up to 2-fold higher in NSCLC compared to controls (FIG. 6). At a cut-off of 118.9 ng/ml, PRO-C19 could discriminate between healthy and NSCLC with an AUROC of 0.823 with a corresponding sensitivity of 82.4% and specificity of 76.7% (Table 5). Comparing individual stages to healthy controls, PRO-C19 was also significantly elevated in stage II (p=0.0011), stage III (p=0.0012) and stage IV (p=0.0041) as compared to healthy controls (FIG. 7). There was a trend towards higher mean PRO-C19 levels in higher stages. To evaluate PRO-C19 as an early-detection marker for NSCLC, diagnostic accuracy for stages I+II was assessed. PRO-C19 could discriminate between healthy and stages I+II NSCLC with an AUROC of 0.762 at a cut-off of 118.9 with a corresponding sensitivity of 70.0% and specificity of 76.7% (Table 5). At a higher specificity of 96.7% the sensitivity dropped to 35%.

Lastly, PRO-C19 was assessed in a separate cancer cohort including 20 pancreatic cancer, colorectal cancer (CRC), kidney cancer, stomach cancer, ovarian cancer, breast cancer, bladder cancer, lung cancer, melanoma, head and neck (H&N) cancer, prostate cancer and lastly 3 liver cancer patients, as well as 33 healthy controls (Table 6). There was no significant different between age or gender between cancers and healthy controls. However, cancer samples were exclusively from Caucasian patients, whereas the healthy controls were a mix of Caucasian, Black and Hispanic ethnicities. There was no significant association of PRO-C19 levels with date of sample collection, gender, age or BMI. Mean PRO-C19 levels were significantly elevated in all cancers compared to controls (FIG. 8.) PRO-C19 was generally excellent at discriminating between healthy and cancer for all cancer types. This is summarized in table 7.

### DISCUSSION

The current study demonstrates the technical validation of an ELISA measuring the C-terminus of type XIX collagen named PRO-C19. PRO-C19 was specific towards the intended epitope and was technically robust. PRO-C19 was assessed in a panel of serum samples from healthy individuals and cancer patients to demonstrate biological relevance and biomarker potential. PRO-C19 levels were significantly elevated in several types of cancer, proved excellent at discriminating between NSCLC and healthy individuals and exhibited moderate diagnostic accuracy in early stages of NSCLC.

In human adults, collagen XIX expression can be very limited as exemplified by the 10-6% of the dry weight of umbilical cord tissue that type XIX collagen amounted to (25). However, a separate study quantifying collagen XIX in different tissue extracts and biological fluids found it detectable in the circulation (26). Based on the data, collagen XIX is released into circulation of healthy adults in modest amounts and circulating collagen XIX levels are significantly increased in some cancer types. Collagen XIX has previously been linked to breast cancer progression. In this context, as the BMZ surrounding breast tumours was broken down during cancer progression, the staining of collagen XIX protein was also lost¹³. Collagen XIX expression is strongly associated with the BMZ in general and the breakdown of the epithelial and vascular BMZ of the breast could lead to the release of type XIX collagen into circulation. Based on the data, an increase in the levels of circulating collagen XIX is also linked to breast cancer. There can be distinct differences in the organization of the BMZ of different tumour types e.g. The epithelial BMZ is broken down around invasive carcinomas of the breast, whereas it can remain intact around invasive glands in colon, prostate and lung epithelial malignancies (13,27). A limitation of this approach to collagen XIX quantification is that the source tissue cannot be determined, although the tissue the tumour is found in is the likely contributor.

Collagen XIX has also been linked to neurodegenerative diseases including amyotrophic lateral sclerosis (ALS) and Parkinson's. Collagen XIX expression is downregulated in the peripheral blood of Parkinson's patients (35). Contrastingly, in ALS collagen XIX increased with progressing disease and increased mortality risk (18,36,37). Therefore, the PRO-C19 assay could also be used to detect and diagnose neurodegenerative diseases including amyotrophic lateral sclerosis (ALS) and Parkinson's disease.

The presence of type XIX collagen in connection to lung cancer has not previously been demonstrated. It has been observed in moderate amounts in the lungs of mice embryos, whereas only trace amounts were seen in adults, which could suggest a developmental role of collagen XIX in the lungs (11). Such an expression pattern is seen in several proteins and pathways important for cancer progression, and indeed several aspects of the developmental process are reactivated during tumorigenesis, including EMT (28-30). Thus, a role in development could hint at a role in cancer as well.

Anti-tumour properties have been assigned to collagen XIX. Interestingly, the NC1 domain can, once cleaved off, inhibit invasion and angiogenesis in melanoma (22). This was demonstrated in an in vivo mouse model where the NC1 (XIX) peptide inhibited tumour growth, and where the NC1 (XIX) peptide inhibited angiogenesis by MMP-14 and VEGF inhibition (20). It was later discovered that NC1 (XIX) signalling is likely mediated by the αvβ3 integrin (31). In a separate study, it was demonstrated that the NC1 (XIX) peptide could promote the formation of inhibitory nerve terminals through α5β1 integrin (23). These integrin receptors are expressed by both epithelial and endothelial lung cells and play a role in NSCLC, so it would be interesting to see the effects of NC1 (XIX) peptides in lung cancer (32-34).

The PRO-C19 assay is not specific towards the neo-epitope generated during plasmin cleavage and subsequent release of the NC1 domain. However, it can quantify any fragment containing the C-terminal epitope. Knowledge of how collagen XIX is cleaved or otherwise processed is lacking, so PRO-C19 could hypothetically measure a large and diverse population of collagen XIX fragments that all contain the C-terminal epitope. Further investigation into how collagen XIX is processed and if any fragments can be quantified in circulation is warranted. A separate assay specific towards the neo-epitope generated during plasmin cleavage could help in this regard.

In terms of diagnostic accuracy, PRO-C19 performed modestly in stages I and II of NSCLC patients. Given the small samples sizes, a relatively wide confidence interval included AUC values ranging from 0.62 corresponding to bad diagnostic performance up to 0.87 corresponding to good performance. A follow-up study to validate and pinpoint the diagnostic accuracy of PRO-C19 is needed. Additionally, at high specificities above 95%, where diagnostic tests are usually most relevant, the sensitivity of PRO-C19 in early stage NSCLC was down to 35%. Future studies should also investigate combining PRO-C19 with other NSCLC biomarkers to improve overall accuracy. Furthermore, future studies into early detection could also assess PRO-C19 in high-risk individuals before an eventual NSCLC diagnosis.

This study has several major limitations: Given the strictly exploratory nature of this study, the use of so-called “samples of convenience” and post-hoc analysis can introduce bias. In numbers, this bias is evidenced by the differences in sample sizes, age, gender and ethnicity of the compared groups. Clinical data of the study participants is also limited, so additional hidden bias could also arise. The results and conclusions of this study are therefore merely our first attempt at probing the biology of collagen XIX in cancer.

In conclusion, an ELISA targeting the C-terminus of type XIX collagen, named PRO-C19, was developed and validated. PRO-C19 was used to quantify collagen XIX in the serum of cancer patients, where it was significantly elevated in all cancer types investigated as compared to healthy controls. PRO-C19 was subsequently assessed in two separate NSCLC cohorts where it was also significantly elevated and exhibited moderate diagnostic accuracy in early stage NSCLC. In all, PRO-C19 and collagen XIX shows potential as a cancer biomarker.

### Tables

TABLE 1
Summary of the technical validation of PRO-C19

| Test | Result |
|---|---|
| IC50 | 29.5 ng/ml |
| Measurement range | 3.31-214 ng/ml |
| Detection range | 1.23-443 ng/ml |
| Minimum required dilution in human serum | 1:4 |
| Dilution recovery of human serum below 1:4 | 102% |
| Spiking recovery of peptide in serum | 119% |
| Interference haemoglobin, low/high | 95.1%/93.4% |
| Interference lipids, low/high | 104%/104% |
| Interference biotin, low/high | 101%/101% |
| Inter-assay variation | 10.9% |
| Intra-assay variation | 6.63% |
| Analyte stability (24 hrs 4° C./4 hrs 20° C.) | 85.3%/82.3% |
| Freeze-thaw stability up to four cycles | 97.7% |

TABLE 2
Demographics of cohort 1

| | Healthy | | | | |
|---|---|---|---|---|---|
| | controls | | Cancers | | p- |
| | (n = 38) | | (n = 75) | | value |
| Age, mean (SD) | 71.3 | (5.78) | 60.33 | (11.4) | <0.0001 |
| Male, n (%) | 0 | (0.00%) | 39 | (52.0%) | <0.0001 |
| Caucasians, n (%) | — | | 73 | (97.3%) | — |
| Cancer type, n | — | | Breast: 12, Colon: 7, | | — |
| | | | Gastric: 9, Melanoma: 6, | | |
| | | | NSCLC: 11, Ovary: 8, | | |
| | | | Pancreas: 2, Prostate: | | |
| | | | 13, SCLC: 7 | | |
| PRO-C19, mean | 32.10 | (18.18) | 58.86 | (42.58) | 0.0004 |
| (SD) | | | | | |

TABLE 3
Demographics of cohort 2

| | Healthy | | | | |
|---|---|---|---|---|---|
| | controls | | NSCLC | | p- |
| | (n = 24) | | (n = 40) | | value |
| Age, mean (SD) | 35.46 | (10.5) | 63.75 | (3.66) | <0.0001 |
| Male, n (%) | 6 | (25.0%) | 21 | (52.5%) | 0.0389 |
| Caucasians, n (%) | 10 | (41.7%) | 40 | (100%) | <0.0001 |
| Tumour stage, n | — | | III: 20, IV: 20 | | — |
| NSCLC subtype, n | — | | AC: 28, SCC: 12 | | — |
| PRO-C19, mean (SD) | 34.55 | (18.47) | 125.1 | (59.38) | <0.0001 |

TABLE 4
Demographics of cohort 3

| | Healthy | | | | |
|---|---|---|---|---|---|
| | controls | | NSCLC | | p- |
| | (n = 30) | | (n = 34) | | value |
| Age, mean (SD) | 49.73 | (14.51) | 62.03 | (1.8) | <0.0001 |
| Male, n (%) | 16 | (53.3%) | 19 | (55.9%) | >0.9999 |
| Caucasians, n (%) | 18 | (60.0%) | 34 | (100%) | <0.0001 |
| Tumour stage, n | — | | I: 10, II: 10, | | — |
| | | | III: 9, IV: 5 | | |
| NSCLC subtype, n | — | | AC: 17, SCC: 17 | | — |
| PRO-C19, mean (SD) | 95.85 | (76.96) | 201.5 | (120.3) | <0.0001 |

TABLE 5
Diagnostic accuracy of PRO-C19

| | AUROC | | Sensitivity, % | Specificity, % | Cut-off |
|---|---|---|---|---|---|
| Test | (95% CI) | p-value | (95% CI) | (95% CI) | (ng/ml) |
| Cohort 1 | 0.995 | <0.0001 | 100 | 94.74 | 63.324 |
| Controls v | (0.918-1.00) | | (71.5-100.0) | (82.3-99.4) | |
| NSCLC | | | | | |
| Cohort 1 | 0.808 | 0.0048 | 71.4 | 84.2 | 54.3 |
| Controls v | (0.663-0.910) | | (29.0-96.3) | (68.7-94.0) | |
| SCLC | | | | | |
| Cohort 1 | 0.814 | <0.0001 | 75.0 | 78.95 | 41.848 |
| Controls v | (0.678-0.910) | | (42.8-94.5) | (62.7-90.4) | |
| breast | | | | | |
| cancer | | | | | |
| Cohort 1 | 0.839 | 0.0003 | 75.0 | 92.1 | 60.308 |
| Controls v | (0.701-0.931) | | (34.9-96.8) | (78.6-98.3) | |
| ovarian | | | | | |
| cancer | | | | | |
| Cohort 2 | 0.980 | <0.0001 | 97.50 | 91.67 | 55.612 |
| Controls v | (0.909-0.999) | | (86.8-99.9) | (73.0-99.0) | |
| NSCLC | | | | | |
| Cohort 3 | 0.823 | <0.0001 | 82.35 | 76.7 | 118.896 |
| Controls v | (0.707-0.907) | | (65.5-93.2) | (57.7-90.1) | |
| NSCLC | | | | | |
| Cohort 3 | 0.762 | 0.0002 | 70.0 | 76.7 | 118.896 |
| Controls v | (0.620-0.871) | | (45.7-88.1) | (57.7-90.1) | |
| Stage I + II | | | | | |
| NSCLC | | | | | |

TABLE 6
Demographics of cohort 4

| | Healthy | | Cancer | | Total | |
|---|---|---|---|---|---|---|
| | (N = 33) | | (N = 223) | | (N = 256) | |
| Diagnosis | | | | | | |
| Healthy | 33 | (100%) | 0 | (0%) | 33 | (12.9%) |
| bladder cancer | 0 | (0%) | 20 | (9.0%) | 20 | (7.8%) |
| breast cancer | 0 | (0%) | 20 | (9.0%) | 20 | (7.8%) |
| CRC | 0 | (0%) | 20 | (9.0%) | 20 | (7.8%) |
| H&N cancer | 0 | (0%) | 20 | (9.0%) | 20 | (7.8%) |
| kidney cancer | 0 | (0%) | 20 | (9.0%) | 20 | (7.8%) |
| liver cancer | 0 | (0%) | 3 | (1.3%) | 3 | (1.2%) |
| lung cancer | 0 | (0%) | 20 | (9.0%) | 20 | (7.8%) |
| melanoma | 0 | (0%) | 20 | (9.0%) | 20 | (7.8%) |
| ovarian cancer | 0 | (0%) | 20 | (9.0%) | 20 | (7.8%) |
| pancreatic cancer | 0 | (0%) | 20 | (9.0%) | 20 | (7.8%) |
| prostate cancer | 0 | (0%) | 20 | (9.0%) | 20 | (7.8%) |
| stomach cancer | 0 | (0%) | 20 | (9.0%) | 20 | (7.8%) |
| Stages | | | | | | |
| I | 0 | (0%) | 7 | (3.1%) | 7 | (2.7%) |
| II | 0 | (0%) | 49 | (22.0%) | 49 | (19.1%) |
| III | 0 | (0%) | 93 | (41.7%) | 93 | (36.3%) |
| IV | 0 | (0%) | 74 | (33.2%) | 74 | (28.9%) |
| Missing | 33 | (100%) | 0 | (0%) | 33 | (12.9%) |
| Age (years) | | | | | | |
| Mean (SD) | 57.7 | (5.69) | 59.3 | (11.2) | 59.1 | (10.7) |
| Median [Min, Max] | 57.0 | [49.0, 69.0] | 61.0 | [30.0, 87.0] | 60.0 | [30.0, 87.0] |
| Missing | 0 | (0%) | 1 | (0.4%) | 1 | (0.4%) |
| Sex | | | | | | |
| Male | 21 | (63.6%) | 121 | (54.3%) | 142 | (55.5%) |
| Female | 12 | (36.4%) | 102 | (45.7%) | 114 | (44.5%) |
| Ethnicity | | | | | | |
| Black | 13 | (39.4%) | 0 | (0%) | 13 | (5.1%) |
| Caucasian | 11 | (33.3%) | 223 | (100%) | 234 | (91.4%) |
| Hispanic | 9 | (27.3%) | 0 | (0%) | 9 | (3.5%) |

CRC: Colorectal cancer
H&N: Head & neck cancer

TABLE 7
Diagnostic accuracy of PRO-C19 in cohort 4

| | AUROC | | Sensitivity, % | Specificity, % | Cut-off |
|---|---|---|---|---|---|
| Test | (95% CI) | p-value | (95% CI) | (95% CI) | (ng/mL) |
| Cohort 4 | 0.947 | <0.0001 | 95.00 | 93.94 | 63.56 |
| Controls v | (0.848-0.990) | | (75.1-99.9) | (79.8-99.3) | |
| bladder | | | | | |
| cancer | | | | | |
| Cohort 4 | 0.915 | <0.0001 | 90.00 | 90.91 | 61.12 |
| Controls v | (0.806-0.974) | | (68.3-98.8) | (75.7-98.1) | |
| breast | | | | | |
| cancer | | | | | |
| Cohort 4 | 0.986 | <0.0001 | 100.00 | 93.94 | 63.56 |
| Controls v | (0.908-1.000) | | (83.2-100.0) | (79.8-99.3) | |
| CRC | | | | | |
| Cohort 4 | 0.979 | <0.0001 | 100.00 | 93.94 | 63.56 |
| Controls v | (0.896-0.999) | | (83.2-100.0) | (79.8-99.3) | |
| H&N | | | | | |
| Cohort 4 | 0.982 | <0.0001 | 100.00 | 93.94 | 63.56 |
| Controls v | (0.900-1.000) | | (83.2-100.0) | (79.8-99.3) | |
| kidney | | | | | |
| cancer | | | | | |
| Cohort 4 | 0.964 | <0.0001 | 95.00 | 93.94 | 63.56 |
| Controls v | (0.872-0.996) | | (75.1-99.9) | (79.8-99.3) | |
| lung cancer | | | | | |
| Cohort 4 | 0.923 | <0.0001 | 95.00 | 90.91 | 61.12 |
| Controls v | (0.817-0.979) | | (75.1-99.9) | (75.7-98.1) | |
| melanoma | | | | | |
| Cohort 4 | 0.985 | <0.0001 | 100.00 | 93.94 | 63.56 |
| Controls v | (0.905-1.000) | | (83.2-100.0) | (79.8-99.3) | |
| ovarian | | | | | |
| cancer | | | | | |
| Cohort 4 | 0.970 | <0.0001 | 95.00 | 93.94 | 63.56 |
| Controls v | (0.881-0.997) | | (75.1-99.9) | (79.8-99.3) | |
| pancreatic | | | | | |
| cancer | | | | | |
| Cohort 4 | 0.941 | <0.0001 | 95.00 | 87.88 | 54.16 |
| Controls v | (0.840-0.987) | | (75.1-99.9) | (71.8-96.6) | |
| prostate | | | | | |
| cancer | | | | | |
| Cohort 4 | 0.941 | <0.0001 | 95.00 | 93.94 | 63.56 |
| Controls v | (0.840-0.987) | | (75.1-99.9) | (79.8-99.3) | |
| stomach | | | | | |
| cancer | | | | | |

### REFERENCES

1. Bray, F. et al. Global cancer statistics 2018: GLOBOCAN estimates of incidence and mortality worldwide for 36 cancers in 185 countries. CA. Cancer J. Clin. 68, 394-424 (2018).
2. Molina, J. R., Yang, P., Cassivi, S. D., Schild, S. E. & Adjei, A. A. Non-small cell lung cancer: epidemiology, risk factors, treatment, and survivorship. Mayo Clin. Proc. 83, 584-94 (2008).
3. Herbst, R. S., Morgensztern, D. & Boshoff, C. The biology and management of non-small cell lung cancer. Nature 553, 446-454 (2018).
4. Siegel, R. L., Miller, K. D. & Jemal, A. Cancer statistics, 2019. CA. Cancer J. Clin. 69, 7-34 (2019).
5. Hanahan, D. & Weinberg, R. A. Hallmarks of Cancer: The Next Generation. Cell 144, 646-674 (2011).
6. Pickup, M. W., Mouw, J. K. & Weaver, V. M. The extracellular matrix modulates the hallmarks of cancer. EMBO Rep. 15, 1243-1253 (2014).
7. Ricard-Blum, S. The collagen family. Cold Spring Harb. Perspect. Biol. 3, a004978 (2011).
8. Yoshioka, H. et al. Synteny between the loci for a novel FACIT-like collagen locus (D6S228E) and α1(IX) collagen (COL9A1) on 6q12-q14 in humans. Genomics 13, 884-886 (1992).
9. Inoguchi, K., Yoshioka, H., Khaleduzzaman, M. & Ninomiya, Y. The mRNA for α1(XIX) Collagen Chain, a New Member of FACITs, Contains a Long Unusual 3′ Untranslated Region and Displays Many Unique Splicing Variants1. J. Biochem. 117, 137-146 (1995).
10. Myers, J. C. et al. The triple-helical region of human type XIX collagen consists of multiple collagenous subdomains and exhibits limited sequence homology to alpha 1(XVI). J. Biol. Chem. 269, 18549-57 (1994).
11. Sumiyoshi, H., Inoguchi, K., Khaleduzzaman, M., Ninomiya, Y. & Yoshioka, H. Ubiquitous Expression of the α1(XIX) Collagen Gene (Col19a1) during Mouse Embryogenesis Becomes Restricted to a Few Tissues in the Adult Organism. J. Biol. Chem. 272, 17104-17111 (1997).
12. Myers, J. C. et al. Biochemical and immunohistochemical characterization of human type XIX defines a novel class of basement membrane zone collagens. Am. J. Pathol. 151, 1729-40 (1997).
13. Amenta, P. S. et al. Loss of types XV and XIX collagen precedes basement membrane invasion in ductal carcinoma of the female breast. J. Pathol. 199, 298-308 (2003).
14. Sumiyoshi, H., Laub, F., Yoshioka, H. & Ramirez, F. Embryonic expression of type XIX collagen is transient and confined to muscle cells. Dev. Dyn. 220, 155-62 (2001).
15. Sumiyoshi, H. et al. Esophageal muscle physiology and morphogenesis require assembly of a collagen XIX-rich basement membrane zone. J. Cell Biol. 166, 591-600 (2004).
16. Su, J., Gorse, K., Ramirez, F. & Fox, M. A. Collagen XIX is expressed by interneurons and contributes to the formation of hippocampal synapses. J. Comp. Neurol. 518, 229-253 (2010).
17. Hilario, J. D., Wang, C. & Beattie, C. E. Collagen XIXa1 is crucial for motor axon navigation at intermediate targets. Development 137, 4261-4269 (2010).
18. Shtilbans, A. et al. Differential gene expression in patients with amyotrophic lateral sclerosis. Amyotroph. Lateral Scler. 12, 250-256 (2011).
19. Calvo, A. C. et al. Collagen XIX Alpha 1 Improves Prognosis in Amyotrophic Lateral Sclerosis. Aging Dis. 10, 278-292 (2019).
20. Ramont, L. et al. The NC1 domain of type XIX collagen inhibits in vivo melanoma growth. Mol. Cancer Ther. 6, 506-514 (2007).
21. Oudart, J.-B. et al. Plasmin releases the anti-tumor peptide from the NC1 domain of collagen XIX. Oncotarget 6, (2015).
22. Oudart, J.-B. et al. The anti-tumor NC1 domain of collagen XIX inhibits the FAK/PI3K/Akt/mTOR signaling pathway through avβ3 integrin interaction. Oncotarget 7, 1516-28 (2016).
23. Su, J. et al. Collagen-derived matricryptins promote inhibitory nerve terminal formation in the developing neocortex. J. Cell Biol. 212, 721-736 (2016).
24. Kehlet, S. N. et al. A fragment of SPARC reflecting increased collagen affinity shows pathological relevance in lung cancer—implications of a new collagen chaperone function of SPARC. Cancer Biol. Ther. 19, 904-912 (2018).
25. Myers, J. C. et al. Type XIX Collagen Purified from Human Umbilical Cord Is Characterized by Multiple Sharp Kinks Delineating Collagenous Subdomains and by Intermolecular Aggregates via Globular, Disulfide-linked, and Heparin-binding Amino Termini. J. Biol. Chem. 278, 32047-32057 (2003).
26. Oudart, J. B. et al. Analytical methods for measuring collagen XIX in human cell cultures, tissue extracts, and biological fluids. Anal. Biochem. 437, 111-117 (2013).
27. Maatta, M., Virtanen, I., Burgeson, R. & Autio-Harmainen, H. Comparative Analysis of the Distribution of Laminin Chains in the Basement Membranes in Some Malignant Epithelial Tumors: The α1 Chain of Laminin Shows a Selected Expression Pattern in Human Carcinomas. J. Histochem. Cytochem. 49, 711-725 (2001).
28. Sedgwick, A. & D'Souza-Schorey, C. Wnt Signaling in Cell Motility and Invasion: Drawing Parallels between Development and Cancer. Cancers (Basel). 8, 80 (2016).
29. Giroux-Leprieur, E., Costantini, A., Ding, V. & He, B. Hedgehog Signaling in Lung Cancer: From Oncogenesis to Cancer Treatment Resistance. Int. J. Mol. Sci. 19, 2835 (2018).
30. Chaffer, C. L., San Juan, B. P., Lim, E. & Weinberg, R. A. EMT, cell plasticity and metastasis. Cancer Metastasis Rev. 35, 645-654 (2016).
31. Oudart, J.-B. et al. The anti-tumor NC1 domain of collagen XIX inhibits the FAK/PI3K/Akt/mTOR signaling pathway through alphavbeta3 integrin interaction. Oncotarget 7, 1516-1528 (2016).
32. Caccavari, F., Valdembri, D., Sandri, C., Bussolino, F. & Serini, G. Integrin signaling and lung cancer. Cell Adh. Migr. 4, 124-129 (2010).
33. Singh, B., Fu, C. & Bhattacharya, J. Vascular expression of the αvβ3-integrin in lung and other organs. Am. J. Physiol. Cell. Mol. Physiol. 278, L217-L226 (2000).
34. Schaffner, F., Ray, A. & Dontenwill, M. Integrin a531, the Fibronectin Receptor, as a Pertinent Therapeutic Target in Solid Tumors. Cancers (Basel). 5, 27-47 (2013).
35. Infante, J. et al. Identification of candidate genes for Parkinson's disease through blood transcriptome analysis in LRRK2-G2019S carriers, idiopathic cases, and controls. Neurobiol. Aging 36, 1105-1109 (2015).
36. Calvo, A. C. et al. Genetic biomarkers for ALS disease in transgenic SOD1(G93A) mice. PLOS One 7, e32632 (2012).
37. Ana, C. C. et al. Collagen XIX Alpha 1 Improves Prognosis in Amyotrophic Lateral Sclerosis. Aging Dis. (2018). doi:10.14336/AD.2018.0917
38. Kabat, E. A., T. T. Wu, H. M. Perry, K. S. Gottesman and C. Foeller (1987), Sequences of Proteins of Immunological Interest, United States Department of Health and Human Services, Bethesda, Md., p. 1

1: A monoclonal antibody that specifically recognises and binds to a peptide having the C-terminus amino acid sequence SHAHQRTGGN (SEQ ID NO: 1).

2: The monoclonal antibody of claim 1, wherein the monoclonal antibody is a monoclonal antibody raised against a synthetic peptide having the C-terminus amino acid sequence SHAHQRTGGN (SEQ ID NO: 1).

3: The monoclonal antibody of claim 1, wherein the antibody does not specifically recognise or bind to a peptide having the C-terminus amino acid sequence SHAHQRTGGNX (SEQ ID NO: 2), wherein X represents any amino acid.

4: The monoclonal antibody of claim 1, wherein the antibody does not specifically recognise or bind to a peptide having the C-terminus amino acid sequence SHAHQRTGGNA (SEQ ID NO: 3).

5: The monoclonal antibody of claim 1, wherein the antibody does not specifically recognise or bind to a peptide having the C-terminus amino acid sequence SHAHQRTGG (SEQ ID NO: 4).

6: The monoclonal antibody of claim 1, wherein the antibody does not specifically recognise or bind to a peptide having the C-terminus amino acid sequence GVAPGIGPGG (SEQ ID NO: 5).

7: A method of immunoassay for detecting type XIX collagen in a human biofluid sample, said method comprising contacting a human biofluid sample with a monoclonal antibody that specifically recognises and binds to a peptide having the C-terminus amino acid sequence SHAHQRTGGN (SEQ ID NO: 1), and detecting binding between the monoclonal antibody and peptides in the sample.

8: A method as claimed in claim 7, wherein the detection is quantitative.

9: A method as claimed in claim 7, wherein the immunoassay is a competitive immunoassay.

10: A method as claimed in claim 7, wherein the monoclonal antibody is a monoclonal antibody raised against a synthetic peptide having the C-terminus amino acid sequence SHAHQRTGGN (SEQ ID NO: 1).

11: A method as claimed in claim 7, wherein the antibody does not specifically recognise or bind to a peptide having the C-terminus amino acid sequence SHAHQRTGGNX (SEQ ID NO: 2), wherein X represents any amino acid.

12: A method as claimed in claim 7, wherein the antibody does not specifically recognise or bind to a peptide having the C-terminus amino acid sequence SHAHQRTGGNA (SEQ ID NO: 3).

13: A method as claimed in claim 7, wherein the antibody does not specifically recognise or bind to a peptide having the C-terminus amino acid sequence SHAHQRTGG (SEQ ID NO: 4).

14: A method as claimed in claim 7, wherein the antibody does not specifically recognise or bind to a peptide having the C-terminus amino acid sequence GVAPGIGPGG (SEQ ID NO: 5).

15: A method as claimed in claim 7, wherein the human biofluid sample is from a human patient having medical signs or symptoms indicative of cancer.

16: A method as claimed in claim 7, wherein the method is an immunoassay method for diagnosing and/or monitoring and/or assessing the likelihood of cancer in a patient, the method comprising contacting a biofluid sample obtained from said patient with the monoclonal antibody, detecting and determining the amount of binding between the monoclonal antibody and peptides in the sample, and correlating said amount of binding with values associated with normal healthy subjects and/or values associated with known disease severity and/or values obtained from said patient at a previous time point.

17: A method as claimed in claim 16 wherein the cancer is breast, lung, colon, colorectal, head and neck, kidney, liver, pancreatic, prostate, gastric, melanoma, bladder or ovarian cancer.

18: An assay kit comprising a monoclonal antibody that specifically recognises and binds to a peptide having the C-terminus amino acid sequence SHAHQRTGGN (SEQ ID NO: 1), and at least one of:
a streptavidin coated well plate;
a N-terminal biotinylated peptide having the C-terminus amino acid sequence SHAHQRTGGN (SEQ ID NO: 1);
a calibrator peptide having the C-terminus amino acid sequence SHAHQRTGGN (SEQ ID NO: 1).

19: The assay kit of claim 18, wherein the monoclonal antibody is a monoclonal antibody raised against a synthetic peptide having the C-terminus amino acid sequence SHAHQRTGGN (SEQ ID NO: 1).

20: The assay kit of claim 18, wherein the antibody does not specifically recognise or bind to a peptide having the C-terminus amino acid sequence SHAHQRTGGNX (SEQ ID NO: 2), wherein X represents any amino acid.

21: The assay kit of claim 18, wherein the antibody does not specifically recognise or bind to a peptide having the C-terminus amino acid sequence SHAHQRTGGNA (SEQ ID NO: 3).

22: The assay kit of claim 18, wherein the antibody does not specifically recognise or bind to a peptide having the C-terminus amino acid sequence SHAHQRTGG (SEQ ID NO: 4).

23: The assay kit of claim 18, wherein the antibody does not specifically recognise or bind to a peptide having the C-terminus amino acid sequence GVAPGIGPGG (SEQ ID NO: 5).
